(19) United States
(12) Patent Application Publication
(10) Pub. No.: US 2009/0099569 A1
(43) Pub. Date: Apr. 16, 2009

(54) Intervertebral implant

(75) Inventor: Jens Beger, Tuttlingen (DE)

Correspondence Address: Lipsitz & McAllister, LLC, 755 MAIN STREET, MONROE, CT, 06468, US

(73) Assignee: Aesculap AG, Tuttlingen (DE)

(21) Appl. No.: 12/286,549

(22) Filed: Sep. 29, 2008

Related U.S. Application Data
(63) Continuation of application No. PCT/EP2007/002676, filed on Mar. 27, 2007.

(30) Foreign Application Priority Data
Apr. 6, 2006 (DE) 10 2006 016 987

(51) Int. Cl. A61B 17/58; A61F 2/44

(52) U.S. Cl. 606/90; 623/17.11; 623/17.16

(57) ABSTRACT

In the case of an intervertebral implant with at least one abutment element forming a vertebral body abutment surface, in which the vertebral body abutment surface comprises at least two relatively movable parts, which in a first insertion position are arranged in relation to one another so that they jointly assume a small cross-section, and in a second implantation position so that the cross-section of the vertebral body abutment surface is increased in size in relation to the insertion position, and with an adjusting device for moving the movable parts from the insertion position into the implantation position, to simplify the adjustment of the movable parts from the insertion position into the implantation position, it is proposed that the adjusting device comprises at least one flexible pull element, which acts on one of the relatively movable parts and when pulled moves this relative to the other part into the implantation position.

This application is a continuation of international application number PCT/EP2007/002676 filed on Mar. 27, 2007.

The present disclosure relates to the subject matter disclosed in international application PCT/EP2007/002676 of Mar. 27, 2007 and German patent application 10 2006 016 987.5 of Apr. 6, 2006, which are incorporated herein by reference in their entirety and for all purposes.

### BACKGROUND OF THE INVENTION

The invention relates to an intervertebral implant with at least one abutment element forming a vertebral body abutment surface, in which the vertebral body abutment surface comprises at least two relatively movable parts, which in a first insertion position are arranged in relation to one another so that they jointly assume a small cross-section, and in a second implantation position so that the cross-section of the vertebral body abutment surface is increased in size in relation to the insertion position, and with an adjusting device for moving the movable parts from the insertion position into the implantation position.

Intervertebral implants should have as large a vertebral body abutment surface as possible that is adapted to the size of the end faces of the vertebral body, against which the intervertebral implant comes into abutment. This reduces the risk of the intervertebral implant collapsing or fusing into the vertebral body.

On the other hand, it is desirable to bring the implant into the intervertebral space through as small an access opening as possible, in particular when inserting the implant through a posterior, transforaminal or lateral access opening.

It is known to configure the abutment surfaces of intervertebral implants in multiple parts for this purpose and insert the different parts into the body in a folded position. After insertion into the body through a relatively small access opening the movable parts can be brought into an opened position, in which a relatively large vertebral body abutment surface is formed, the cross-section of which is significantly larger than the cross-section of the access opening into the body (WO 2004/103226 A2). To bring the movable parts into the folded-out position with this known device, either complicated adjusting means with threaded spindles are necessary, or this folding-out must be performed using special instruments, which the surgeon must insert through the access opening into the body and with which he must perform the movement of the movable parts. With the relatively small access openings this is extremely difficult and also hazardous, since relatively high forces may have to be transferred during the opening movement and because vulnerable body parts are situated in the direct surroundings of the intervertebral space.

It is an object of the invention to configure an intervertebral implant of the above-mentioned type so that the movement of the movable parts from the insertion position into the implantation position is simplified.

### SUMMARY OF THE INVENTION

This object is achieved according to the invention with the intervertebral implant of the type described above in that the adjusting device comprises at least one flexible pull element, which acts on one of the relatively movable parts and when pulled moves this relative to the other part into the implantation position. Therefore, it is sufficient to pull on this flexible pull element, which can have a thread-like configuration, to perform the opening movement, in which case relatively high forces can be readily transferred and, moreover, practically no additional structural volume is necessary, and the risk of injury in the surroundings of the operating site is eliminated.

It is beneficial if the flexible pull element is of such a length that one end remains outside the body after insertion of the intervertebral implant into the body, so that a pulling force can be exerted on the flexible pull element by means of this extracorporeal end. Thus, the operating surgeon can grasp the free end of the flexible pull element outside the body and from there cause the movable parts to move into the implantation position by pulling.

The flexible pull element can be displaceably guided on the other part, e.g. through eyelets or other thread guides known per se.

It is particularly advantageous if the flexible pull element is guided on the other part through a deflection means, which changes the direction of the flexible pull element. As a result of this, it is possible to transfer the pulling force to the movable part in the desired direction, while the free end of the pull element can still run in the direction of the insertion opening of the body in a user-friendly manner. In this way, pulling forces can also be transferred in directions, in which an application of force using an instrument would be impossible because of the narrow access opening.

After the operation, the flexible pull element can be detached from the implant by cutting, for example. However, it is provided in a preferred embodiment that the flexible pull element is detachably connected to the part moved by pulling, wherein the detachment only occurs after a specific tension value is exceeded. A predetermined breaking point enables the force necessary to move the movable part into the implantation position to be firstly transferred without problem, and as soon as this has occurred, the operating surgeon can detach the flexible pull element from the moved part by increasing the pulling force and remove it from the body.

The flexible pull element can be a surgical thread, for example.

It is particularly advantageous if the flexible pull element is composed of a material which is resorbable in the body. In such a case, the pull element may also remain in the body, if necessary.

A plurality of pull elements can act on a movable part. These are then directed jointly out of the operation access opening to the outside preferably in the form of thread bundles.

It can be provided that an abutment element has a plurality of movable parts, and that each movable part has at least one associated pull element. In this case, the operating surgeon can also operate a plurality of pull elements simultaneously by pulling on a thread bundle and can thus move a plurality of movable parts simultaneously into the implantation position.

In a preferred embodiment, a locking device is provided, which becomes effective during movement of the movable part as soon as the movable part has reached the implantation position and secures the movable part in relation to the other part. As soon as the implantation position has been reached by pulling on the flexible pull element, the locking device engages and prevents the movable part from moving back into the insertion position. In this instant, the operating surgeon can discontinue the pulling force on the flexible pull element and the movable parts of the abutment element will still remain in the implantation position once this has been reached.

For example, the locking device can have a locking face provided with notches on one of the two parts and a locking member pressed elastically against the locking face on the other of the two parts, which slide along one another during movement of the parts into implantation position. In this case, a relative movement is readily possible in one direction, but is prevented in the opposite direction by engagement of the locking member into the notches of the locking face.

In another configuration it can be provided that the locking device comprises elastically displaceable locking elements, which are disposed on one of the two parts and which in the implantation position engage into a recess on the other of the two parts in a resilient and positive-locking manner.

A fixture of the movable parts in the implantation position can also be achieved if movable support members are arranged on one part of the two parts that are movable into a position supporting the other of the two parts as soon as the other of the two parts has reached the implantation position.

In particular, the support member can be at least one lever arm, which is rotatably disposed on one of the two-parts and which is rotatable into a position supporting the other of the two parts.

It is advantageous in this case if for rotation of the lever arm a threaded spindle is arranged on the part supporting the lever arm.

The above-described locking devices and arrangements for fixing the movable parts in the implantation position can also be used in intervertebral implants, in which the movable parts are not brought into the implantation position by means of flexible pull elements, but in a different manner, e.g. by means of inserted instruments, by means of filled expansion bodies or by means of a swellable material, which actuates the movement of the movable parts into the implantation position by liquid absorption and increase in volume.

It is favourable if at least one stop is provided, which restricts the movement of the movable part into the implantation position. The implantation position is thus precisely defined, and therefore the operating surgeon does not need to check precisely what position the movable parts occupy, but can pull until the implantation position is reached.

The movable parts of the abutment element can be very different in configuration and in this regard reference is made, inter alia, to the different configurations described in WO 2004/103226 A2.

Thus, the abutment element can have, for example, at least two plate-like abutment members, which can be pivoted relative to one another and can be pivoted apart for transition into the implantation position.

The intervertebral implant comprises at least one such abutment element, but an abutment element of this type, which can be changed in its cross-sectional size, is preferably arranged respectively on the upper and on the lower end of the intervertebral implant.

In a particularly preferred embodiment, swellable material is arranged on the side of the abutment element remote from the vertebral body abutment surface and undergoes an increase in volume upon liquid absorption and thus presses the parts of the abutment element into the implantation position. As a result of this, the movable parts are secured in the implantation position and this fixture can either be provided alone or in support of a locking device, which holds the movable parts in the implantation position. However, a consideration when using a swellable material of this type is that the increase in volume as result of liquid absorption that occurs during implantation can take several hours, so that it is beneficial to combine the holding forces of the swellable material with such a locking device, which at least at the beginning of the swelling process assumes the task of securing the movable parts in the implantation position.

In particular, it can be provided that the swellable material forms a swellable core between the two abutment elements of the intervertebral implant. This swellable material then has the function of a cushion between the abutment elements, which allows a certain mobility of the two abutment elements relative to one another and the restoring forces of which are determined, inter alia, by the swelling behaviour, as is also the case with a natural intervertebral disc.

The following description of preferred embodiments of the invention serves as more detailed explanation in association with the drawing.

### BRIEF DESCRIPTION OF THE DRAWINGS

FIG. 1 is a perspective view of an intervertebral implant on a vertebral body with the movable parts in the insertion position;

FIG. 2 is a view similar to FIG. 1 with the movable parts in the implantation position and the intervertebral implant between two vertebral bodies;

FIG. 3 is a side view of the implant of FIG. 1 in the insertion position;

FIG. 4 is a front view of the implant in the insertion position;

FIG. 5 is a plan view onto the implant of FIG. 1 in the implantation position;

FIG. 6 is a perspective view of the two components of an intervertebral implant with a lever arrangement for securing the movable parts in the implantation position;

FIG. 7 is a sectional view taken along line 7-7 in FIG. 6;

FIG. 8 is a view similar to FIG. 7 in a modified exemplary embodiment with a single-arm lever as support member;

FIG. 9 is a view in partial section taken along line 9-9 in FIG. 10 of the hinge region of an intervertebral implant with a locking device for fixture of the movable parts in the implantation position;

FIG. 10 is a plan view in partial section onto the hinge region of the intervertebral implant of FIG. 9; and

FIG. 11 is a view similar to FIG. 5 with a locking device with locking pins for fixture of the movable parts in the implantation position.

### DETAILED DESCRIPTION OF THE INVENTION

The intervertebral implant 1 shown in the drawing is inserted between two vertebral bodies 3, 4 during implantation into the intervertebral space 2 and there replaces the intervertebral disc removed from the intervertebral space 2. On its underside and on its upper side the intervertebral implant 1 shown in the drawing comprises a respective abutment element 5 and 6, which are both identical in configuration, but arranged mirror-inverted to one another. Only one of the two abutment elements will be explained in more detail below.

The abutment element 5 has a central, plate-like support section 7 with a rectangular cross-section, on the longitudinal sides of which a plate-like pivot part 8, 9 is respectively disposed to pivot around a pivot axis running along the longitudinal edges. One pivot part has the shape of a sector of a circle with an arc-shaped outer edge 10, the other pivot part 9 is substantially rectangular, but the outer edge 11 remote from the pivot axis is curved slightly inwards and merges at its ends into transversely running edges 13, 14 via rounded corners 12. When the two pivot parts 8, 9 are pivoted into the plane of the support section 7, a plane abutment surface 15, which is composed of the individual faces of the support section 7 and the two pivot parts 8, 9 and is delimited on opposing longitudinal sides in an arc shape in the same direction, results in this way on the outer surface of the respective abutment element 5. Thus, this abutment element 15 is adapted to the contour of the end faces of the two vertebral bodies 3, 4 and can be selected to be of such a size that it substantially abuts against the entire front surface of the vertebral bodies 3, 4.

In a modified embodiment the two pivot parts 8, 9 could also be inclined slightly relative to the central support section 7, so that optimum adaptation to the respective geometry of the vertebral front surface can be achieved. In this case, there results an abutment surface of the implant that is not plane in the entire region, but has regions with slightly different inclinations.

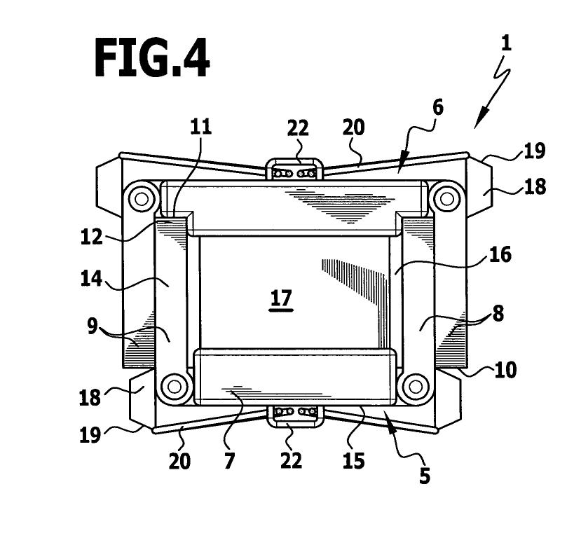

The pivot axes of the two abutment elements 5, 6 have a spacing from one another that differs slightly between the two abutment elements 5, 6, so that the pivot parts 8, 9 of the two abutment elements 5, 6 lie flat one on top of the other (FIG. 4) when they are folded over at right angles to the support section 7. This position of the pivot parts 8, 9 where they are pivoted 90° is referred to as the insertion position and the pivoted-out position, in which the pivot parts 8, 9 run in the plane of the support section 7, is referred to as the implantation position.

In the insertion position, the two pivot parts 8, 9 project into the interstice between the two abutment elements 5, 6 and delimit this on its longitudinal sides. In the shown exemplary embodiment, the remaining interstice 16 between the two abutment elements 5, 6 is filled by a core 17 composed of a swellable material, which abuts against the two support sections 7 on the inside. The material of this core has the ability to increase in volume upon liquid absorption. The increase in volume can amount to as much as six-times the initial volume without liquid absorption. In principle, all non-degradable hydrophilic polymers are conceivable as materials in this case. Examples are polyacrylic acid and its derivatives such as polymethacrylic acid, polyacrylamide, polyacrylonitrile, polyacrylate, polyhydroxy ethyl methacrylates, or also other substances such as e.g. polyvinylpyrrolidone (PVP), polyurethanes, high-molecular polyvinyl alcohol.

Also conceivable are polymer blends (copolymers linked to one another through bonds) composed of the abovementioned polymers or interpenetrating networks (IPNs) composed of the abovementioned polymers. IPNs consist of at least two different polymers, the polymer chains of which are entangled and are linked to one another by means of physical interactions (van der Waals, electrostatic, H-bridge bonds and/or ionic forces).

Further polymer mixtures that can be used are copolymers and also IPNs of polyacrylates (polyacrylic acid and its derivatives such as polymethacrylic acid, polyacrylamide, polyacrylonitrile, polyacrylate) with polycaprolactone.

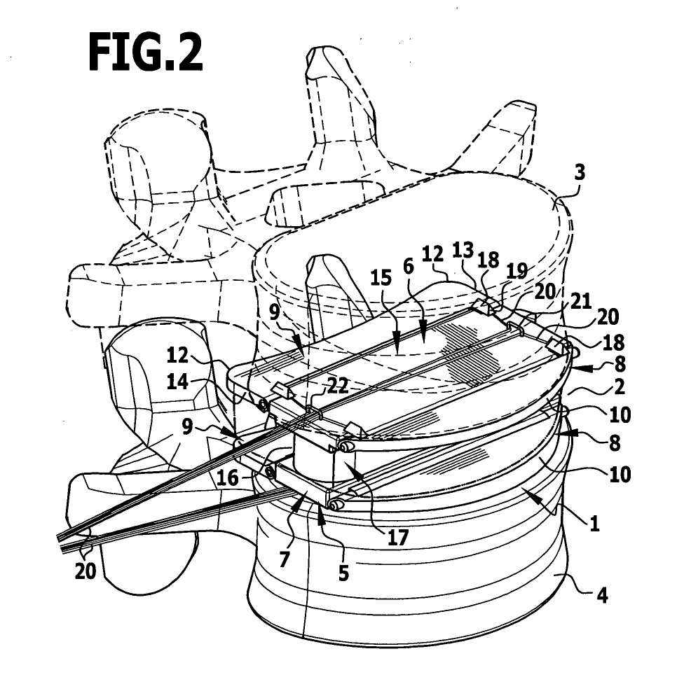

As can be seen from the illustration in FIG. 4, the cross-section of the intervertebral implant 1 is substantially smaller in the insertion position, i.e. when the pivot parts 8, 9 are folded, than in the implantation position, in which the pivot parts 8, 9 are pivoted into the plane of the support section 7 (FIG. 2).

In order to perform this pivoting movement, lugs 18 projecting upwards beyond the pivot axes are arranged on the pivot parts 8, 9 in the region of the pivot axes, and a pull thread 20 respectively acts on the free ends 19 of these lugs that project beyond the pivot axis and extends away from these engagement points on the upper side of the support sections 7 parallel thereto and transversely to the pivot axis of the pivot parts 8, 9. In the exemplary embodiment shown in the drawing, each pivot part 8 bears two such lugs 18, so that two pull threads 20 act on each pivot part 8, 9. All pull threads 20 are guided on the upper side of the support sections 7 through deflection eyelets 21, 22, which are arranged on the longitudinal centre axis of the support sections 7 and enable the pull threads 20 to be deflected so that they run along the longitudinal centre axis of the support section 7.

Moreover, the lugs 18 also act as a stop, by means of which the outward pivoting movement of the pivot parts 8, 9 is restricted as soon as the implantation position is reached, i.e. as soon as the pivot parts 8, 9 stand in the same plane as the support section 7. Finally, the lugs 18 also form projections on the abutment surface, which penetrate into the substance of the abutting vertebral bone and therefore act as ribs or spikes in conventional implants, the position of the implant relative to the vertebral body being fixed by these projections.

The pull threads 20 can be surgical threads, for example, a material which is resorbable in the body being advantageously used. Polyglycolic acid, poly-p-dioxanone, copolymers of glycolic acid and/or trimethyl carbonate and/or caprolactone and/or p-dioxanone and/or lactic acid, for example, can be used as material for such a resorbable suture material. These substances can be used in different proportions by weight and in a wide variety of combinations.

To implant the intervertebral implant 1, the pivot parts 8, 9 are firstly brought into the insertion position, as shown in FIG. 4. In this folded state, the intervertebral implant has a relatively small cross-section and can therefore also be inserted into the body through small access openings. The free ends of the pull threads 20 remain outside the body during insertion.

After insertion of the intervertebral implant, the operating surgeon can pivot the pivot parts 8, 9 from the folded insertion position into the pivoted-out implantation position by pulling on the pull threads 20, and this movement is achieved solely by pulling on the pull threads 20 and, if necessary, by holding these pulling forces at the support sections 7. A suitable instrument, which applies the holding forces while also guiding the thread ends, can be used for this.

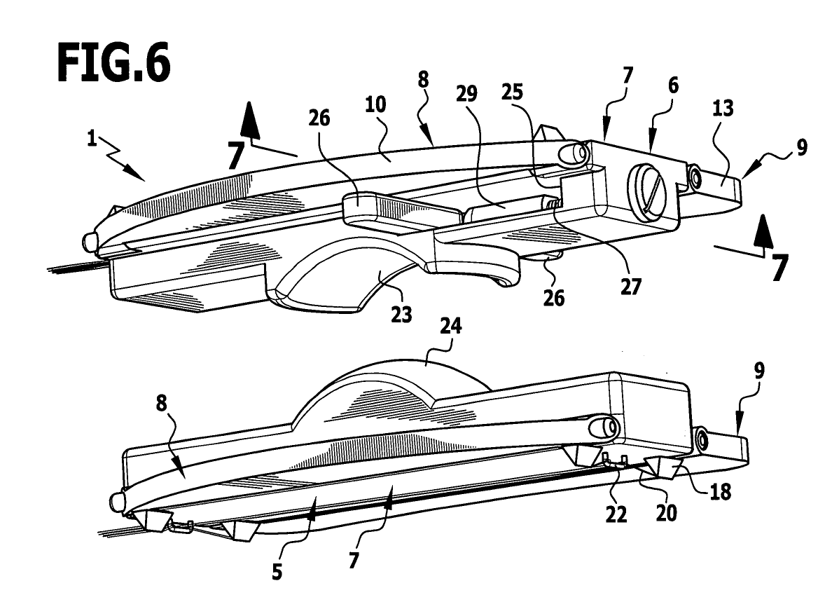

The pivot parts 8, 9 must be secured in the pivoted-out implantation position, so that they can perform their support function and not pivot back into the folded up position again. This can be achieved in a wide variety of ways, as will be explained below with reference to FIGS. 6 to 11.

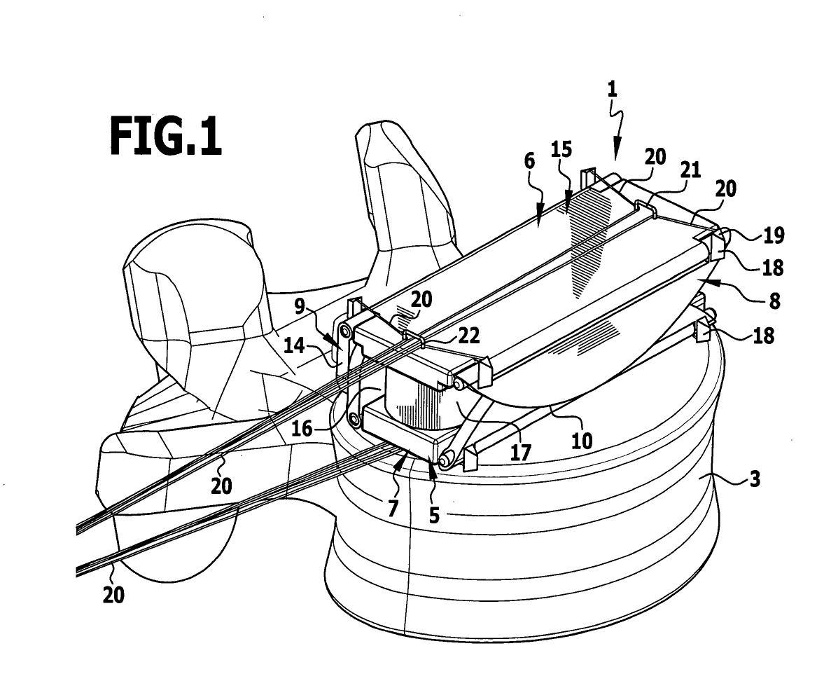

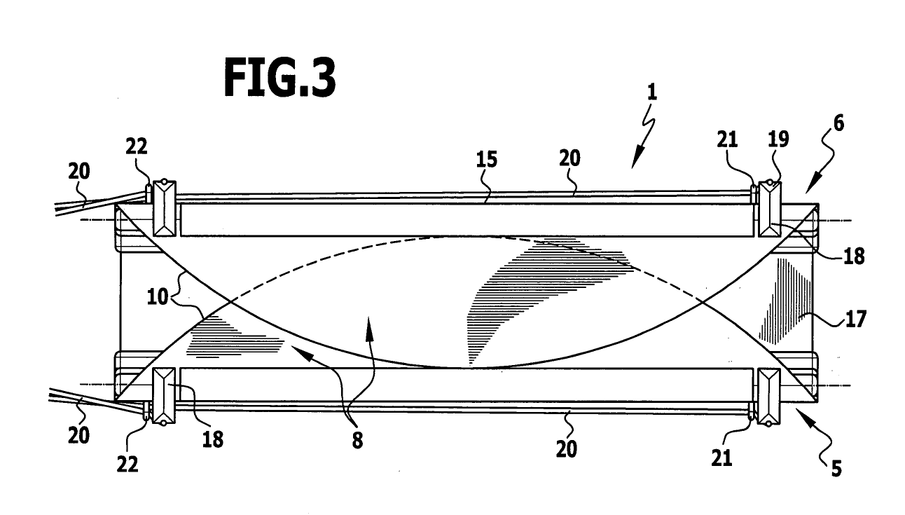

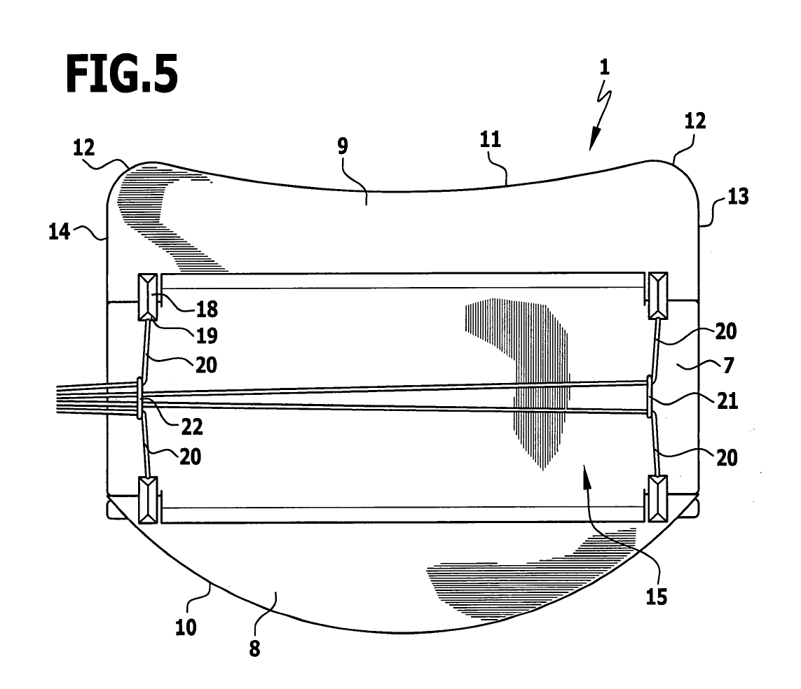

An intervertebral implant is described in FIG. 6 that differs from the intervertebral implant of FIGS. 1 to 5, amongst other things, in that the lower abutment element 5 and the upper abutment element 6 are not connected to one another by means of a swellable core 17, but by means of a spherical bearing shell 23 and a bearing projection 24 engaging into this and complementary to the bearing shell 23. The two abutment elements 5, 6 can thus be pivoted to a small extent relative to one another, but are secured against lateral displacement. Otherwise, a similar structure of the abutment elements 5, 6 is selected. All configurations of the intervertebral implant can either be configured with a swellable core or with a bearing shell and a bearing projection in the described manner, and other connections of the two abutment elements 5, 6 are also fundamentally possible.

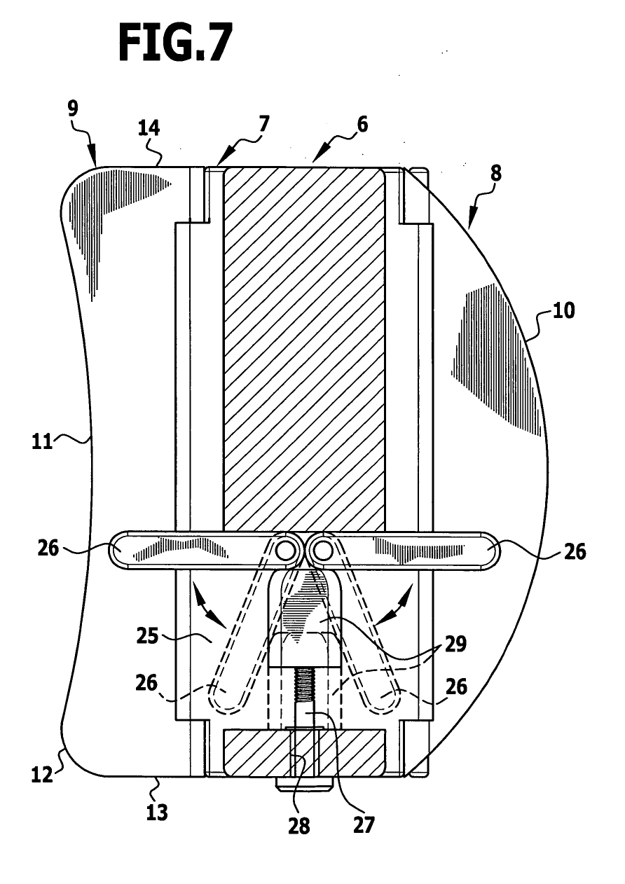

In the exemplary embodiment of FIG. 6, two levers 26 are disposed in a receiving chamber 25 below the central support section 7 to pivot around a rotational axis running perpendicularly to the abutment elements 5, 6. In the insertion position both levers 26 are pivoted fully into the receiving chamber 25, but can thus be pivoted out of the receiving chamber 25 so that they project laterally beyond the contour of the support section 7. To pivot the levers 26, a threaded spindle 27 is rotatably disposed in a threaded bore 28, which bears a pressing body 29 on its free end that pivots the two levers 26 outwards when advancing towards the two levers 26 that are pivoted into the receiving chamber 25 (FIG. 7). The levers 26 are pivoted outwards as soon as the pivot parts 8, 9 have reached the implantation position, and the pivoted-out levers 26 then abut against the underside of the two pivoted-out pivot parts 8, 9 and support these, so that the pivot parts 8, 9 can no longer pivot back into the folded position.

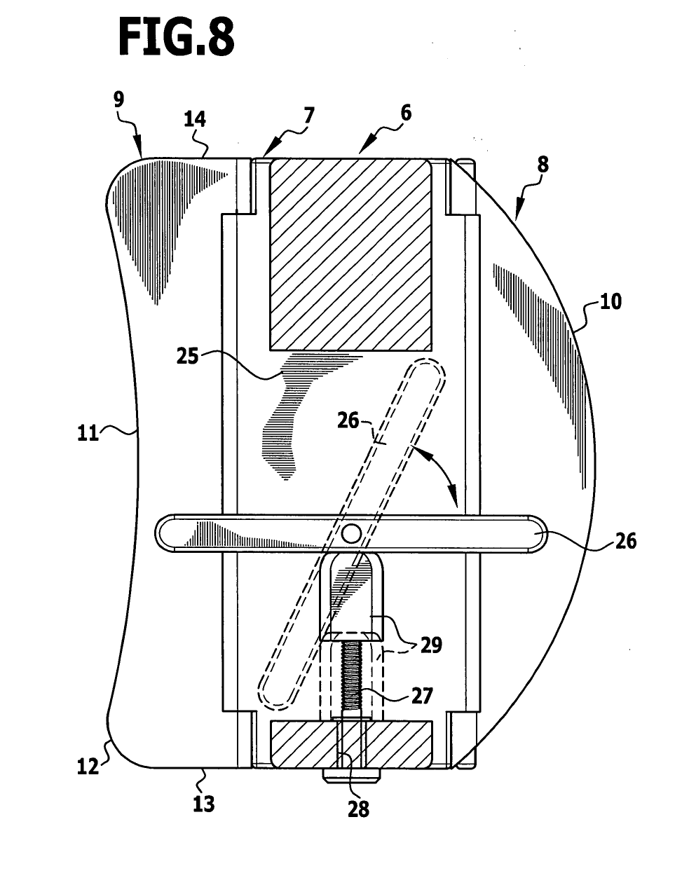

A very similar structure is selected in the exemplary embodiment of FIG. 8, and instead of the two levers 26 that can be pivoted into the receiving chamber 25, only a single lever 26 is provided, which in the pivoted-out state projects out of the receiving chamber 25 to both sides and thus supports the two pivot parts 8, 9 simultaneously in the implantation position.

In the exemplary embodiment of FIGS. 6 to 8, it is necessary to pivot the levers 26 by means of the threaded spindle 27.

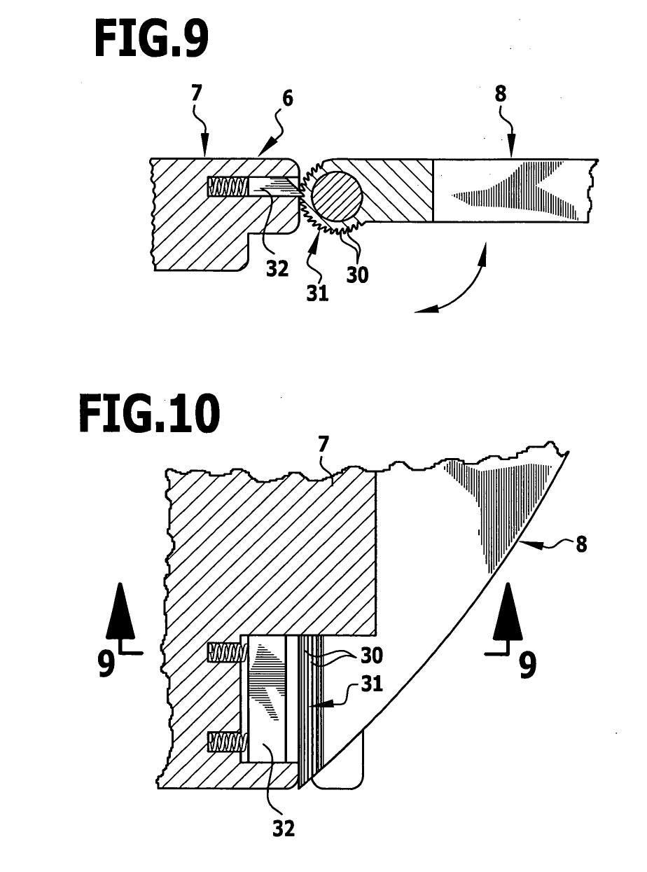

In the exemplary embodiments of FIGS. 9 to 11, there results an automatic locking of the pivot parts 8, 9 in the implantation position. In the exemplary embodiment of FIGS. 9 and 10, the pivot parts are fitted on the outside in the region of the pivot mounting with a locking face 31 provided with notches 30 and spring-loaded locking elements 32, which are displaceable transversely to the pivot axis, abut against the locking face 31 and slide along the locking faces 31 during pivoting of the pivot parts 8, 9, are disposed in the support sections 7. In this case, the geometry of the notches 30 and the locking elements 32 is selected so that the parts can slide along one another in one direction, while in the opposite direction locking occurs by engagement of the locking element 32 into the notches 30 and the pivot part 8, 9 is thus locked in position. In other words, each pivot part 8, 9 can only be pivoted out of the insertion position into the implantation position and not in the reverse direction.

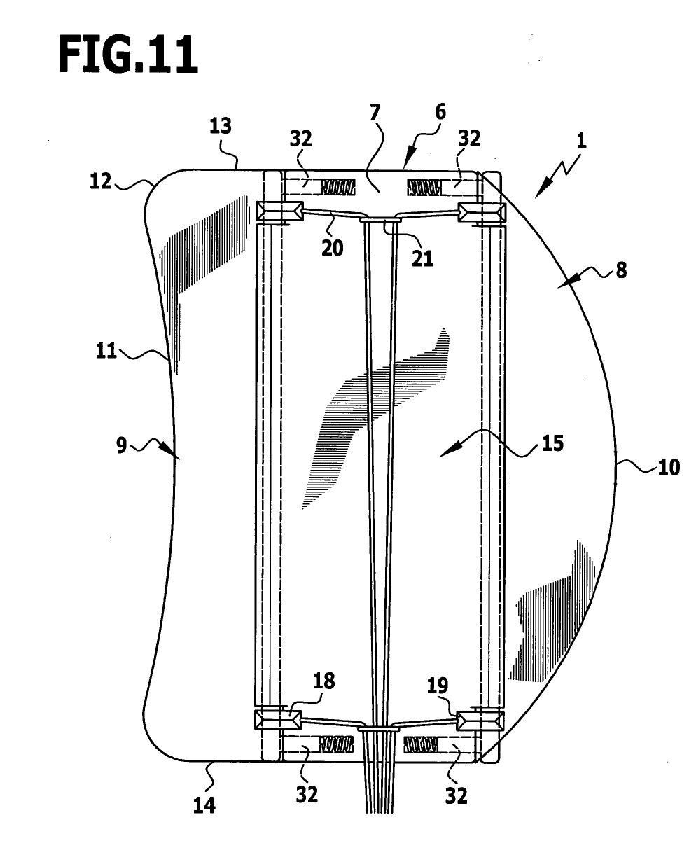

In the exemplary embodiment of FIG. 11, pin-type locking elements 32 are disposed to be elastically displaceable in the support section 7 in a similar manner and engage in recesses of the pivot parts 8, 9 in a positive-locking arrangement as soon as these have reached the pivoted-out implantation position, so that the pivot parts 8, 9 are thus fixed in the pivoted-out implantation position.

After the pivot parts 8, 9 are pivoted out into the implantation position, the pull threads 20 are no longer needed and can be cut off or broken off with a powerful pull. It is advantageous in this case if the pull threads 20 are only held at the lugs 18 with a force that is less than the tearing strength of the pull threads 20, so that with a powerful pull the pull threads 20 can be broken off at the connection points to the lugs 18 in a defined manner and thus completely removed.

As soon as the implant is located in the body, it comes into contact with body fluid, and this causes the core 17 to increase in volume by swelling. It completely fills the interstice 16 and also expands laterally, wherein the material of the core 17 not only abuts against the inside of the support section 7, but it also abuts against the underside of the two pivot parts 8, 9 and thus presses these into the pivoted-out implantation position. A locking device that holds the pivot parts 8, 9 in the implantation position is thus assisted and, if necessary, in the absence of such a locking device, the core 17 can press the pivot parts 8, 9 permanently into the implantation position as a result of these forces and thus against the front surfaces of the two abutting vertebral bodies 3, 4.

1. Intervertebral implant with at least one abutment element forming a vertebral body abutment surface, in which the vertebral body abutment surface comprises at least two relatively movable parts, which in a first insertion position are arranged in relation to one another so that they jointly assume a small cross-section, and in a second implantation position so that the cross-section of the vertebral body abutment surface is increased in size in relation to the insertion position, and with an adjusting device for moving the movable parts from the insertion position into the implantation position, wherein the adjusting device comprises at least one flexible pull element, which acts on one of the relatively movable parts and when pulled moves this relative to the other part into the implantation position.

2. Intervertebral implant according to claim 1, wherein the flexible pull element is of such a length that one end remains outside the body after insertion of the intervertebral implant into the body, so that a pulling force can be exerted on the flexible pull element by means of this extracorporeal end.

3. Intervertebral implant according to claim 1, wherein the flexible pull element is displaceably guided on the other part.

4. Intervertebral implant according to claim 3, wherein the flexible pull element is guided on the other part through a deflection means, which changes the direction of the flexible pull element.

5. Intervertebral implant according to claim 1, wherein the flexible pull element is detachably connected to the part movable by pulling, wherein the detachment only occurs after a specific tension value is exceeded.

6. Intervertebral implant according to claim 1, wherein the flexible pull element is a surgical thread.

7. Intervertebral implant according to claim 1, wherein the flexible pull element is composed of a material which is resorbable in the body.

8. Intervertebral implant according to claim 1, wherein a plurality of pull elements act on a movable part.

9. Intervertebral implant according to claim 1, wherein an abutment element has a plurality of movable parts, and that each movable part has at least one associated pull element.

10. Intervertebral implant according to claim 1, wherein a locking device is provided, which becomes effective during movement of the movable part as soon as the movable part has reached the implantation position and secures the movable part in relation to the other part.

11. Intervertebral implant according to claim 10, wherein the locking device has a locking face provided with notches on one of the two parts and a locking member pressed elastically against the locking face on the other of the two parts, which slide along one another during movement of the movable parts into implantation position.

12. Intervertebral implant according to claim 10, wherein the locking device comprises elastically displaceable locking elements, which are disposed on one of the two parts and which in the implantation position engage into a recess on the other of the two parts in a resilient and positive-locking manner.

13. Intervertebral implant according to claim 1, wherein movable support members are arranged on one part of the two parts and are movable into a position supporting the other of the two parts as soon as the other of the two parts has reached the implantation position.

14. Intervertebral implant according to claim 13, wherein the support member is at least one lever arm, which is rotatably disposed on one of the two parts and which is rotatable into a position supporting the other of the two parts.

15. Intervertebral implant according to claim 14, wherein for rotation of the lever arm a threaded spindle is arranged on the part supporting the lever arm.

16. Intervertebral implant according to claim 1, wherein at least one stop is provided, which restricts the movement of the movable part into the implantation position.

17. Intervertebral implant according to claim 1, wherein the abutment element has at least two plate-like abutment members, which can be pivoted relative to one another and can be pivoted apart for transition into the implantation position.

18. Intervertebral implant according to claim 1, wherein an abutment element, which can be changed in its cross-sectional size, is arranged respectively on the upper and on the lower end of the intervertebral implant.

19. Intervertebral implant according to claim 1, wherein swellable material is arranged on the side of the abutment element remote from the vertebral body abutment surface and undergoes an increase in volume upon liquid absorption and thus presses the parts of the abutment element into the implantation position.

20. Intervertebral implant according to claim 19, wherein the swellable material forms a swellable core between the two abutment elements.
